(19) United States
(12) Patent Application Publication
(10) Pub. No.: US 2023/0153684 A1
(43) Pub. Date: May 18, 2023

(54) LEARNING DEVICE, LEARNING METHOD, AND MEASUREMENT DEVICE

(71) Applicant: KABUSHIKI KAISHA TOKAI RIKA DENKI SEISAKUSHO, Aichi (JP)

(72) Inventors: Tetsuya HIROTA, Aichi (JP); Daisuke KAWAMURA, Aichi (JP); Ryugo FUJITA, Aichi (JP)

(73) Assignee: KABUSHIKI KAISHA TOKAI RIKA DENKI SEISAKUSHO, Aichi (JP)

(21) Appl. No.: 17/917,393

(22) PCT Filed: Feb. 4, 2021
(86) PCT No.: PCT/JP2021/004094

§ 371 Date: Oct. 6, 2022

(30) Foreign Application Priority Data
Apr. 20, 2020 (JP) 2020-074590

(51) Int. Cl. G06N 20/00

(52) U.S. Cl. CPC G06N 20/00

(57) ABSTRACT

To more efficiently acquire vital data which is less affected by noise.

Provided is a learning device including a learning unit that performs learning related to an output of vital data indicating vital signs of a subject by using first sensor data acquired from the subject through a first method as learning data and using training data based on second sensor data acquired from the subject in the same period as a period of acquisition of the first sensor data through a second method which is less affected by noise than the first method, wherein the learning unit performs learning further on the basis of third sensor data which is acquired in the same period as the period of acquisition of the first sensor data and the second sensor data and available as an index indicating a magnitude of influence of the noise occurring in the first sensor data.

### TECHNICAL FIELD

The present invention relates to a learning device, a learning method, and a measurement device.

### BACKGROUND ART

In recent years, various devices that acquire vital data of subjects have been developed. For example, Patent Literature 1 discloses a technique of measuring an electrocardiographic waveform of a subject using electrodes provided on a seat and a steering wheel of a moving object. According to the technique, it is possible to reduce the burden on the subject accompanying the acquisition of the electrocardiographic waveform.

### CITATION LIST

### Patent Literature

Patent Literature 1: JP 2009-142575A

### SUMMARY OF INVENTION

### Technical Problem

However, in the technique described in Patent Literature 1, noise is likely to occur due to vibration of a moving object, body movement of a subject, or the like, and thus there is the possibility of a decrease in the accuracy of acquisition of an electrocardiographic waveform.

Consequently, the present invention was contrived in view of the above problem, and an object of the present invention is to provide a mechanism that makes it possible to more efficiently acquire vital data which is less affected by noise.

### Solution to Problem

In order to solve the above problem, according to a certain aspect of the present invention, there is provided a learning device including a learning unit that performs learning related to an output of vital data indicating vital signs of a subject by using first sensor data acquired from the subject through a first method as learning data and using training data based on second sensor data acquired from the subject in the same period as a period of acquisition of the first sensor data through a second method which is less affected by noise than the first method, wherein the learning unit performs learning further on the basis of third sensor data which is acquired in the same period as the period of acquisition of the first sensor data and the second sensor data and available as an index indicating a magnitude of influence of the noise occurring in the first sensor data.

In addition, in order to solve the above problem, according to another aspect of the present invention, there is provided a learning method including performing learning related to an output of vital data indicating vital signs of a subject by using first sensor data acquired from the subject through a first method as learning data and using training data based on second sensor data acquired from the subject in the same period as a period of acquisition of the first sensor data through a second method which is less affected by noise than the first method, wherein the learning further includes performing learning further on the basis of third sensor data which is acquired in the same period as the period of acquisition of the first sensor data and the second sensor data and available as an index indicating a magnitude of influence of the noise occurring in the first sensor data.

In addition, in order to solve the above problem, according to another aspect of the present invention, there is provided a measurement device including a measurement unit that outputs vital data indicating vital signs of a subject using first sensor data acquired from the subject through a first method as an input, wherein the measurement unit outputs the vital data by using the first sensor data as learning data, using training data based on second sensor data acquired from the subject in the same period as a period of acquisition of the first sensor data through a second method which is less affected by noise than the first method, and using a trained model having performed learning related to an output of the vital data further on the basis of third sensor data which is acquired in the same period as the period of acquisition of the first sensor data and the second sensor data and available as an index indicating a magnitude of influence of the noise occurring in the first sensor data.

### Advantageous Effects of Invention

As described above, according to the present invention, it is possible to provide a mechanism that makes it possible to more efficiently acquire vital data which is less affected by noise.

### BRIEF DESCRIPTION OF DRAWINGS

FIG. 1 is a diagram illustrating a functional configuration example of a learning device 10 according to an embodiment of the present invention.

FIG. 2 is a diagram illustrating a functional configuration example of a measurement device 20 according to the same embodiment.

FIG. 3 is a diagram illustrating an example of a general electrocardiographic waveform in one period.

FIG. 4 is a diagram illustrating an example of learning data and training data according to an embodiment of the present invention.

FIG. 5 is a diagram illustrating an example of input and output of a measurement unit 220 according to the same embodiment.

FIG. 6 is a diagram illustrating an example of input and output of the measurement unit 220 according to the same embodiment.

FIG. 7 is a diagram for explaining an example of a case where the accuracy of vital data to be output decreases due to the influence of noise contained in first sensor data according to the same embodiment.

FIG. 8 is a diagram for explaining learning based on third sensor data according to the same embodiment.

FIG. 9 is a flowchart illustrating a flow of a learning phase according to the same embodiment.

FIG. 10 is a flowchart illustrating a flow of a measurement phase according to the same embodiment.

### DESCRIPTION OF EMBODIMENTS

Hereinafter, preferred embodiments of the present invention will be described in detail with reference to the accompanying drawings. It should be noted that, in this specification and the accompanying drawings, components that have substantially the same functional configurations are denoted with the same reference numerals, and repeated explanation thereof is omitted.

### <Configuration Example>

### (Learning Device 10)

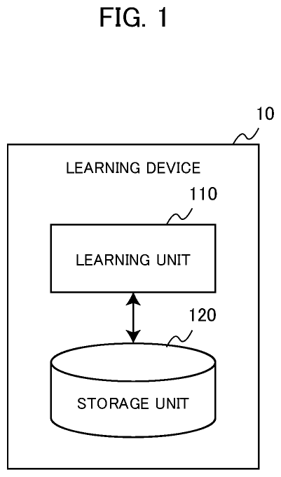

A learning device 10 according to the present embodiment may be a device that performs supervised learning using at least the same type of sensor data acquired in the same period through two different methods as an input. Here, the term “supervised learning” refers to a method of imparting a set of input data (learning data) and correct data (training data) for the input data to a computer and causing the computer to learn a correspondence between the two. FIG. 1 is a diagram illustrating a functional configuration example of the learning device 10 according to the present embodiment. As shown in FIG. 1, the learning device 10 according to the present embodiment may include a learning unit 110 and a storage unit 120. Meanwhile, hereinafter, a case where the learning device 10 performs learning related to an output of vital data indicating vital signs of a subject will be described as an example.

The learning unit 110 according to the present embodiment may perform learning related to an output of vital data, for example, by using first sensor data acquired from a subject through a first method as learning data and using second sensor data acquired from the subject in the same period as the period of acquisition of the first sensor data through a second method which is less affected by noise than the first method as training data. According to such a configuration, by learning a correspondence relation between the first sensor data containing much noise and the second sensor data which is less affected by noise, it is possible to generate a trained model that outputs vital data with noise removed from the first sensor data.

In addition, the learning unit 110 according to the present embodiment may perform learning further based on third sensor data which is acquired in the same period as the period of acquisition of the first sensor data and the second sensor data and available as an index indicating the magnitude of influence of the noise occurring in the first sensor data. The learning based on the third sensor data according to the present embodiment will be described in detail separately.

The learning unit 110 according to the present embodiment may perform the learning as described above using any machine learning method that makes it possible to realize supervised learning. The learning unit 110 performs learning using algorithms such as, for example, a neural network and a support vector machine (SVM).

The function of the learning unit 110 is realized by a processor such as, for example, a graphics processing unit (GPU). The details of the function of the learning unit 110 according to the present embodiment will be described in detail separately.

The storage unit 120 according to the present embodiment stores various types of information relating to the operation of the learning device 10. The storage unit 120 stores, for example, first sensor data, second sensor data, third sensor data, various parameters, and the like used for learning of the learning unit 110.

Hereinbefore, the functional configuration example of the learning device 10 according to the present embodiment has been described. Meanwhile, the above configuration described with reference to FIG. 1 is merely an example, and the configuration of the learning device 10 according to the present embodiment is not limited to such an example. The learning device 10 according to the present embodiment may further include, for example, an operation unit that accepts an operation performed by an operator, an output unit for outputting various types of data, and the like. The configuration of the learning device 10 according to the present embodiment can be flexibly modified according to specifications and operations.

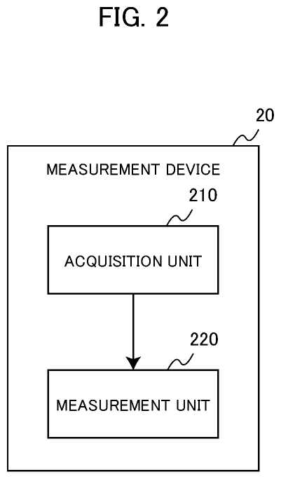

Subsequently, a functional configuration example of a measurement device 20 according to the present embodiment will be described. The measurement device 20 according to the present embodiment may be a device that measures vital data using a trained model constructed by the learning device 10. FIG. 2 is a diagram illustrating a functional configuration example of the measurement device 20 according to the present embodiment. As shown in FIG. 2, the measurement device 20 according to the present embodiment may include an acquisition unit 210 and a measurement unit 220.

The acquisition unit 210 according to the present embodiment acquires first sensor data from a subject. For this purpose, the acquisition unit 210 according to the present embodiment includes various sensors according to the characteristics of the first sensor data to be acquired. In addition, the acquisition unit 210 according to the present embodiment acquires third sensor data. For this purpose, the acquisition unit 210 according to the present embodiment includes various sensors according to the characteristics of the third sensor data to be acquired. For example, an example of the third sensor data according to the present embodiment is acceleration data with a subject or a device predicted to come into contact with the subject as a detection target. In this case, the acquisition unit 210 may include an acceleration sensor.

The measurement unit 220 according to the present embodiment outputs vital data indicating vital signs of a subject using the first sensor data and the third sensor data acquired by the acquisition unit 210 as an input. In this case, the measurement unit 220 according to the present embodiment outputs vital data using a trained model constructed through learning performed by the learning unit 110. For example, the measurement unit 220 according to the present embodiment may output the vital data by using the first sensor data as learning data, using training data based on the second sensor data acquired from the subject in the same period as the first sensor data through a second method which is less affected by noise than the first method, and using a trained model having performed learning related to an output of the vital data.

According to the above configuration, it is possible to obtain high-accuracy vital data from which the influence of noise is removed using only the first sensor data into which noise is assumed to be mixed. Meanwhile, the function of the measurement unit 220 according to the present embodiment is realized by various processors.

In addition, the measurement unit 220 according to the present embodiment may output the vital data using a trained model having performed learning related to an output of the vital data further on the basis of the third sensor data in addition to the first sensor data and the second sensor data. The effect of the learning based on the third sensor data according to the present embodiment will be described in detail separately.

Hereinbefore, the functional configuration example of the measurement device 20 according to the present embodiment has been described. Meanwhile, the above configuration described with reference to FIG. 2 is merely an example, and the functional configuration of the measurement device 20 according to the present embodiment is not limited to such an example. The measurement device 20 according to the present embodiment may further include an operation unit, an output unit, an analysis unit that analyzes vital data, a notification unit that performs various notifications on the basis of the analysis results, and the like. The configuration of the measurement device 20 according to the present embodiment can be flexibly modified according to the characteristics of vital data which is a measurement target, the usage application of the vital data, and the like.

### <Details>

Next, the sensor data according to the present embodiment will be described with specific examples. In recent years, devices that acquire various types of sensor data have been developed. In addition, even in a case where the same type of sensor data is acquired, there may be a plurality of methods. Here, a case where a change in voltage caused by cardiac activity of a subject is acquired as an electrocardiographic waveform is assumed.

Examples of methods of acquiring an electrocardiographic waveform include a 12-lead electrocardiogram method of directly attaching a plurality of electrodes to the skin of a subject and recording a change in voltage using the plurality of electrodes, and the like. According to such a method, it is possible to obtain a high-accuracy electrocardiographic waveform which is less affected by noise. On the other hand, such a method often restricts the behavior of a subject, and may cause the subject to feel annoyed because the electrodes are directly attached to the skin.

In addition, another method of acquiring an electrocardiographic waveform is to install electrodes at a plurality of locations expected to come into contact with a subject and record a change in voltage obtained when the subject touches the plurality of electrodes. Such a method is used, for example, in a case where the electrocardiographic waveform of a subject who operates the device is desired to be acquired, or the like. As an example, a technique of disposing electrodes on a steering wheel, a driver's seat, and the like with which a driver who drives a moving object such as a vehicle is expected to come into contact during driving and acquiring the driver's electrocardiogram is known. According to such a technique, since there is no need to attach the electrodes directly to the skin of the driver, it is possible to acquire an electrocardiographic waveform without the driver's awareness. On the other hand, in this case, noise is likely to occur due to body movement of the driver accompanying driving behavior, vibration of the vehicle, or the like, and thus there is the possibility of a decrease in the accuracy of the acquired electrocardiographic waveform.

In this way, while each of a plurality of methods of acquiring sensor data has advantages, there are also cases where a difference occurs in the accuracy of the sensor data to be acquired. Therefore, there is a demand for a technique of simultaneously improving the accuracy of acquisition of sensor data while making use of the advantages of a certain method.

In order to solve the above point, the learning unit 110 according to the present embodiment may perform learning, for example, by using first sensor data obtained through a first method as learning data and using training data based on second sensor data acquired in the same period as the first sensor data through a second method which is less affected by noise than the first method. According to this, it is also possible to construct a trained model that outputs high-accuracy vital data which is less affected by noise from only the first sensor data.

Hereinafter, a case where the vital data according to the present embodiment is data related to cardiac activity will be described an example. In this case, the learning unit 110 may learn an output of data related to the activity of the subject's heart to be inspected using a first electrocardiographic waveform acquired through the first method as learning data and using training data based on a second electrocardiographic waveform acquired in the same period as the first electrocardiographic waveform through the second method.

In this case, the above first method may be a method of acquiring an electrocardiographic waveform using at least two electrodes expected to come into contact with the subject, and the above second method may be a method of acquiring an electrocardiographic waveform using at least two electrodes attached directly to the skin of the subject.

For example, in a case where the subject is a driver who drives a moving object such as a vehicle, the two electrodes used in the above first method may be provided on a seat on which the subject sits and an operated device (for example, a steering wheel) which is operated by the subject.

According to the configuration as described above, while maintaining the advantages of the first method such as not causing the driver to feel annoyed, it is possible to acquire high-accuracy data obtained by eliminating noise caused by body movement of the driver, vibration of the vehicle, or the like.

Meanwhile, the learning unit 110 according to the present embodiment may perform learning related to an output of a corrected electrocardiographic waveform obtained by removing noise from the first electrocardiographic waveform using the second electrocardiographic waveform itself as training data. In this case, it is possible to obtain various physiological indexes by analyzing the corrected electrocardiographic waveform according to the purpose.

On the other hand, in a case where a physiological index desired to be obtained from the electrocardiographic waveform is determined in advance, it is also possible to cause the learning unit 110 to learn a specified feature point corresponding to the physiological index. Here, a feature point (characteristic waveform) in a general electrocardiographic waveform will be described.

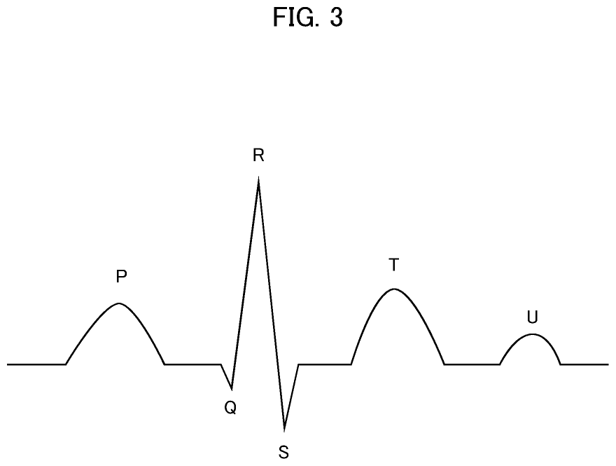

FIG. 3 is a diagram illustrating an example of a general electrocardiographic waveform in one period. Meanwhile, in FIG. 3, the horizontal axis represents the elapse of time, and the vertical axis represents a change in voltage. As shown in FIG. 3, a plurality of characteristic waveforms exhibiting characteristic shapes can be observed in a general electrocardiographic waveform. Examples of the characteristic waveforms include a P wave, a Q wave, an R wave, an S wave, a QRS wave (formed from a Q wave, an R wave, and an S wave), a T wave, a U wave, and the like.

Among these waves, for example, the R wave is a characteristic waveform which is important as an index of heart rate variability (fluctuation). An interval between an R wave in a certain period and an R wave in the next period (RRI: R-R interval) is used to calculate the period of the heartrate. In addition, RRI is also known to cause fluctuation due to stress or fatigue, and is an effective physiological index at the time of detecting the physical burden or psychological burden on a subject. Besides, for example, a Q-T interval (QTI) which is an interval between a Q wave and a T wave in one period indicates the time from the beginning of ventricular excitation to the disappearance of excitation, and is an important physiological index for the detection of arrhythmia or the like.

From this, the learning unit 110 according to the present embodiment may perform learning related to an output of existence probability data indicating the existence probability of the above specified feature point in the first electrocardiographic waveform using existence probability data indicating the existence probability of feature points in the second electrocardiographic waveform, obtained from the second electrocardiographic waveform, as training data.

The learning unit 110 according to the present embodiment may perform learning related to an output of existence probability data indicating the existence probability of an R wave in the first electrocardiographic waveform, for example, using existence probability data indicating the existence probability of the R wave in the second electrocardiographic waveform as training data.

According to the learning as described above, for example, it is possible to construct a trained model that detects any feature point such as the R wave with a high degree of accuracy. In addition, by using the trained model, it is possible to measure the physiological index of a subject such as RRI in real time.

In this way, the learning unit 110 according to the present embodiment may perform learning using training data according to the usage application of the measurement device 20 in which the trained model is mounted.

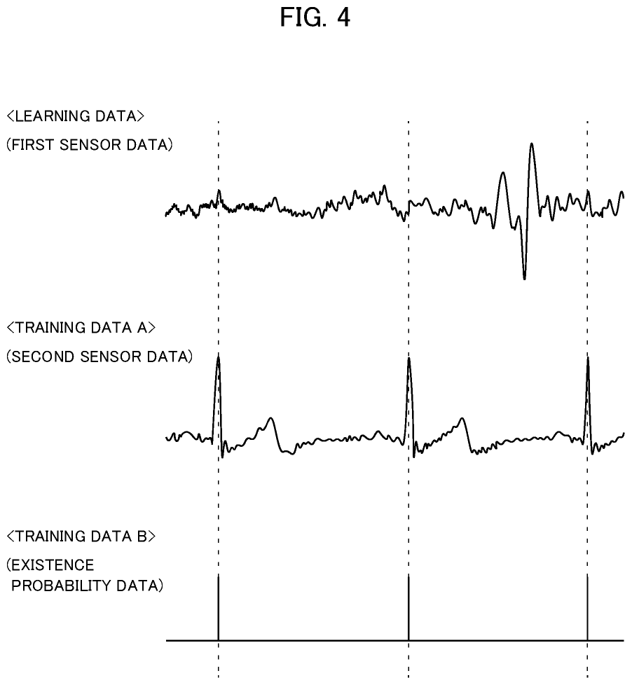

FIG. 4 is a diagram illustrating an example of learning data and training data according to the present embodiment. The upper part of FIG. 4 shows the first sensor data (first electrocardiographic waveform) used as learning data. In addition, the middle part of FIG. 4 shows the second sensor data (second electrocardiographic waveform) acquired in the same period as the first sensor data which is used as training data A. In addition, the lower part of FIG. 4 shows existence probability data of the R wave generated on the basis of the above second sensor data which is used as training data B. Meanwhile, in FIG. 4, the position of the R wave (R-wave peak) in each piece of data is shown by a dotted line.

As shown in FIG. 4, the first sensor data which is acquired through the first method contains much noise, and may not be able to detect the R wave as it is. In this case, by using the second sensor data which is less affected by noise as the training data A, it is possible to cause the learning unit 110 to learn a correspondence relation between the first sensor data and the second sensor data.

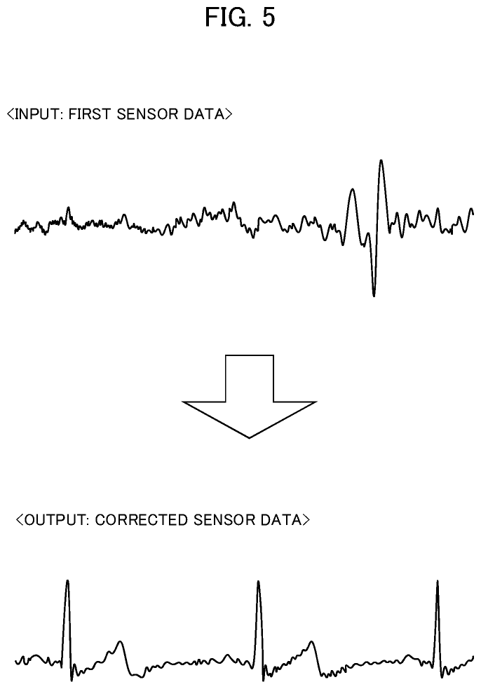

By using the trained model constructed through learning as described above, the measurement unit 220 according to the present embodiment can output corrected sensor data (corrected electrocardiographic waveform) from which noise has been eliminated using the first sensor data as an input as shown in FIG. 5. According to this, by performing any processing or analysis on the output corrected sensor data, it is possible to obtain various physiological indexes of a subject with a high degree of accuracy.

On the other hand, in a case where the existence probability data as shown in FIG. 4 is used as the training data B, it is possible to cause the learning unit 110 to directly learn a correspondence relation between the first sensor data and any feature point.

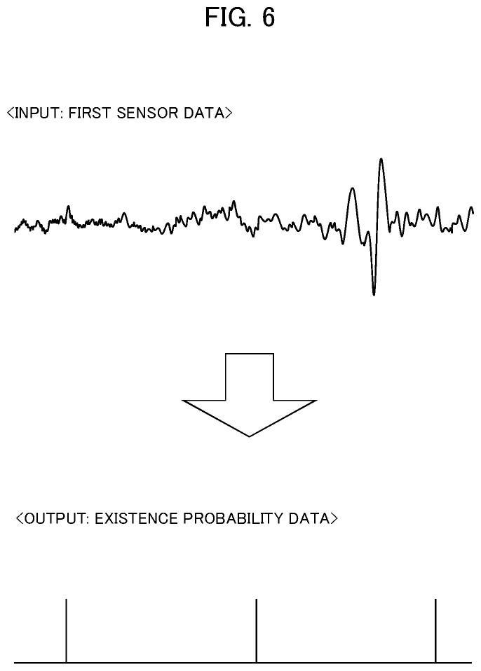

In this case, as shown in FIG. 6, the measurement unit 220 according to the present embodiment can output existence probability data related to a specified feature point such as, for example, the R wave using the first sensor data as an input. According to this, for example, it is possible to measure a physiological index such as RRI in real time and to perform various actions according to the measured value. Meanwhile, in FIGS. 4 and 5, a case where the existence probability data takes a binary value of 0 (not existing) or 1 (existing) has been described above, but the existence probability data according to the present embodiment may take a ternary value or more.

Next, the learning based on the third sensor data according to the present embodiment will be described. As described above, the learning device 10 according to the present embodiment can perform learning related to an output of the vital data by using the first sensor data as learning data to perform learning using training data based on the second sensor data. In addition, the measurement device 20 according to the present embodiment may output the vital data using a trained model having performed learning as described above.

However, here, in a case where the intensity of noise contained in the first sensor data is extremely high, or the like, the learning device 10 cannot correctly learn the correspondence between the first sensor data and the second sensor data, and as a result, there is also the possibility of a decrease in the accuracy of the vital data which is output by the measurement device 20.

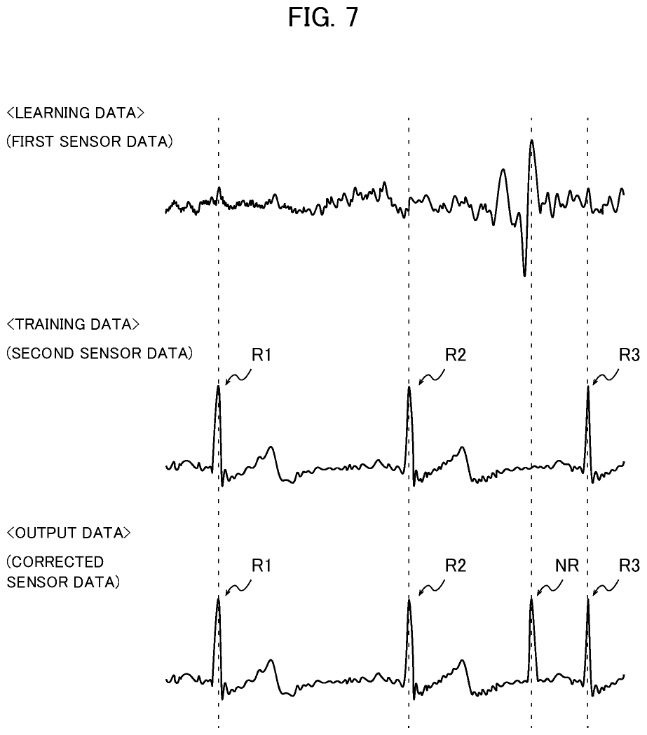

FIG. 7 is a diagram for explaining an example of a case where the accuracy of vital data to be output decreases due to the influence of noise contained in the first sensor data. Meanwhile, FIG. 7 illustrates a case where the learning unit 110 learns the correspondence between the first sensor data and the second sensor data using the first sensor data as learning data and using the second sensor data as training data, and outputs corrected sensor data.

In the present example, the second sensor data includes three R-wave peaks R1 to R3, whereas in the output corrected sensor data, a pseudo R-wave peak NR is erroneously detected at a point in time when the R-wave peak does not exist in the second sensor data in addition to the R-wave peaks R1 to R3.

This is predicted to be due to the high intensity of noise contained in the first sensor data around the point in time. In this way, the intensity of noise contained in the first sensor data can strongly affect the accuracy of detection of various feature points in output data.

For this purpose, the learning unit 110 according to the present embodiment may perform learning further based on the third sensor data acquired in the same period as the period of acquisition of the two pieces of sensor data in addition to the first sensor data and the second sensor data. Here, the third sensor data according to the present embodiment may be various types of sensor data available as an index indicating the magnitude of influence of the noise occurring in the first sensor data.

That is, the learning unit 110 according to the present embodiment can learn the magnitude of the influence of the noise occurring in the first sensor data by using the third sensor data as learning data in addition to the first sensor data.

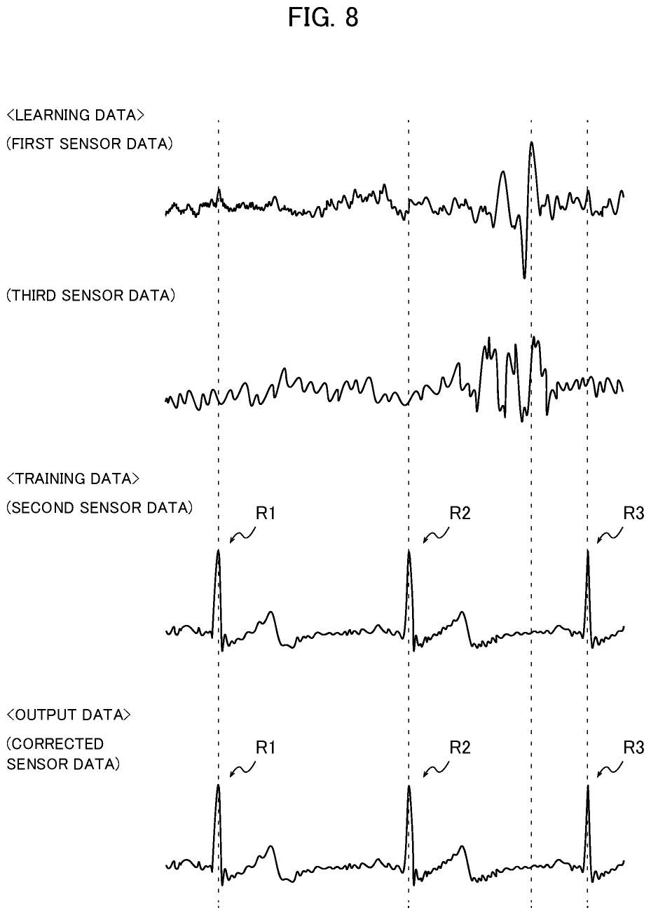

FIG. 8 is a diagram for explaining learning based on the third sensor data according to the present embodiment. Meanwhile, FIG. 8 illustrates a case where the learning unit 110 outputs corrected sensor data using the first sensor data and the third sensor data as learning data and using the second sensor data as training data.

For example, a case where the first sensor data is acquired using electrodes disposed on a seat on which a subject on board a moving object sits and an operated device such as a steering wheel operated by the subject is assumed. In this case, in a situation such as when the contact between the subject and the electrode is temporarily released, it is expected that the intensity of the noise occurring the first sensor data will increase.

Examples of the situations include a situation in which strong vibration occurs in the moving object due to a traveling route or traveling conditions. For this, the third sensor data according to the present embodiment may include, for example, acceleration data acquired with a subject or a device predicted to come into contact with the subject (such as, for example, a seat, a steering wheel, or a moving object) as a detection target. In this case, the learning unit 110 according to the present embodiment may perform learning based on acceleration data as the third sensor data.

In addition, in a situation where the contact between the subject and the electrode is temporarily released, it is assumed that vibration particularly in the direction of gravity (hereinafter also referred to as a z-axis direction) is strongly affected.

Therefore, the learning unit 110 according to the present embodiment may perform learning based on at least acceleration data in the direction of gravity as the third sensor data.

The learning unit 110 according to the present embodiment may perform, for example, learning using only acceleration data in the z-axis direction as the third sensor data, or may perform learning using acceleration data in each of the z-axis direction, the y-axis direction, and the x-axis direction as the third sensor data. In addition, as the third sensor data according to the present embodiment, for example, the norm of acceleration data in the three-axis directions or the jerk in each axial direction (also referred to as acceleration or jerk) may be adopted.

Meanwhile, the acceleration data may be acquired by an acceleration sensor included in a moving object, or may be calculated on the basis of the speed acquired by a speedometer.

According to the learning based on the third sensor data as described above, by canceling the influence of the noise occurring in the first sensor data, it is possible to prevent the pseudo R-wave peak NR or the like from being erroneously detected as shown in the lower part of FIG. 8. In this manner, according to the learning based on the third sensor data according to the present embodiment, it is possible to generate a trained model with further improved accuracy of detection of the vital data than in a case where only the first sensor data and second sensor data are used.

Meanwhile, the third sensor data according to the present embodiment is not limited to acceleration data or data obtained by processing the acceleration data. The third sensor data according to the present embodiment can be various types of data capable of being used to predict a situation in which the contact between the subject and the electrode is temporarily released.

The third sensor data according to the present embodiment may be, for example, angular velocity data, geomagnetic data, or image data. Examples of the image data include data obtained by capturing an image of the state of a traveling route, data obtained by capturing an image of a subject, and the like. Each piece of data as stated above can be used to predict the occurrence of vibration.

In addition, the third sensor data according to the present embodiment may be operation information relating to air conditioning equipment or audio equipment mounted in a moving object. According to such data, it is possible to predict, for example, that the fingers of a subject are temporarily separated from the electrodes disposed on the steering wheel.

### <Flow of Learning Phase and Measurement Phase>

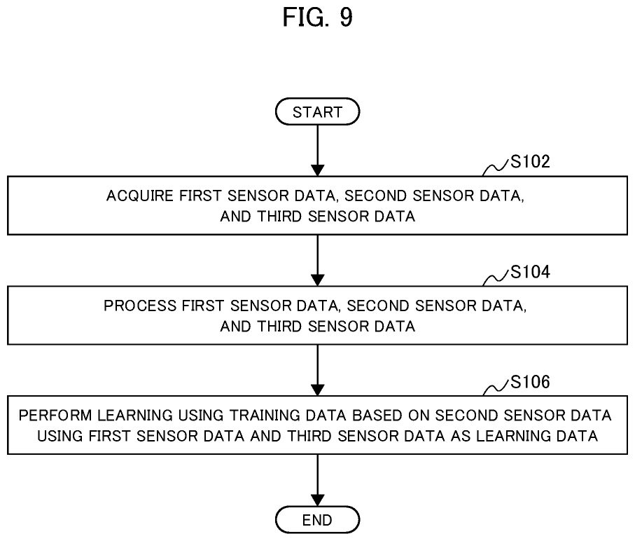

Next, a flow of a learning phase in which learning using the learning device 10 according to the present embodiment is performed and a measurement phase in which measurement using the measurement device 20 is performed will be described. FIG. 9 is a flowchart illustrating a flow of a learning phase according to the present embodiment.

As shown in FIG. 9, in the learning phase according to the present embodiment, the first sensor data, the second sensor data, and the third sensor data are first acquired (S102). In this case, the first sensor data, the second sensor data, and the third sensor data may be acquired together with information such as time stamps to enable synchronization on the time axis. In addition, the first sensor data, the second sensor data, and the third sensor data may be acquired by a device separate from the learning device 10. The acquired first sensor data, second sensor data, and third sensor data are stored in the storage unit 120 of the learning device 10.

Next, the first sensor data, the second sensor data, and the third sensor data are processed as necessary (S104). For example, in a case where the existence probability data related to a specified feature point is used as training data, in step S104, a process of converting the second sensor data acquired in step S102 into the existence probability data is performed. In addition, various filtering processes and the like for reducing noise contained in the first sensor data, the second sensor data, and the third sensor data may be performed. Meanwhile, the processing as described above may be executed by a device separate from the learning device 10.

Next, the learning unit 110 performs learning using training data based on the second sensor data using the first sensor data and the third sensor data as learning data (S106). In this case, the learning unit 110 may use the second sensor data itself (or the second sensor data on which a filtering process has been performed) as training data, or may use the existence probability data generated in step S104 as training data.

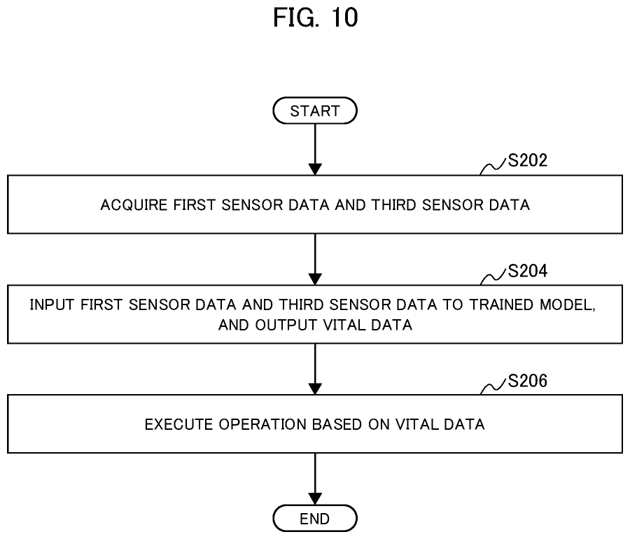

Hereinbefore, the flow of the learning phase according to the present embodiment has been described. Subsequently, a flow of a measurement phase according to the present embodiment will be described. FIG. 10 is a flowchart illustrating a flow of a measurement phase according to the present embodiment.

As shown in FIG. 10, in the measurement phase according to the present embodiment, the acquisition unit 210 first acquires the first sensor data and the third sensor data through the first method (S202). The acquisition unit 210 may acquire, for example, the electrocardiographic waveform of a driver as the first sensor data using a plurality of electrodes disposed on the steering wheel and seat of a vehicle. In addition, the acquisition unit 210 may acquire, for example, acceleration data as the third sensor data using an acceleration sensor disposed on the steering wheel or the like.

Next, the measurement unit 220 inputs the first sensor data and the third sensor data acquired in step S202 as trained model, and outputs the vital data (S204). In a case where learning is performed using the second sensor data as training data in the learning phase, the above vital data can be corrected sensor data obtained by removing noise from the first sensor data. On the other hand, in a case where learning is performed using the existence probability data as training data in the learning phase, the above vital data can be existence probability data indicating the existence probability of any feature point.

Next, various operations based on the vital data output in step S204 are executed as necessary (S206). The above operations may be, for example, notification based on RRI detected from the vital data. The above operations may be executed by a device separate from the measurement device 20.

### <Supplement>

Heretofore, preferred embodiments of the present invention have been described in detail with reference to the accompanying drawings, but the present invention is not limited thereto. It should be understood by those skilled in the art that various changes and alterations may be made without departing from the spirit and scope of the appended claims, and that these changes and alterations also naturally fall within the technical scope of the present invention.

For example, in the above-described embodiment, a case where the learning unit 110 performs learning related to an output of vital data indicating vital signs of a subject has been described as a main example. On the other hand, the target of learning performed by the learning unit 110 is not limited to the output of vital data. The learning unit 110 can also perform learning related to an output of, for example, data indicating the operation status of any device.

In addition, in the above-described embodiment, as the first method of acquiring an electrocardiographic waveform, a method of directly attaching the electrode to the skin of a subject is given as an example with a method of disposing the electrode at a location expected to be touched by the subject as the second method. On the other hand, the first method and the second method in the present technique may be any different methods having a difference in a tendency to be affected by noise. For example, in a case where the heartrate is acquired, the first method may be a non-contact method using a Doppler sensor, and the second method may be a contact method of attaching an electrode to the skin of a subject.

In addition, a series of processes performed by each device described in this specification may be realized using any of software, hardware, and a combination of software and hardware. Programs constituting software are stored in advance, for example, in a recording medium (non-transitory media) provided inside or outside each device. Each program is read, for example, into a RAM when executed by a computer, and is executed by a processor such as a CPU. The above recording medium is, for example, a magnetic disc, an optical disc, a magnetooptic disc, a flash memory, or the like. In addition, the above computer program may be delivered, for example, through a network without using a recording medium.

### REFERENCE SIGNS LIST

10 learning device
110 learning unit
120 storage unit
20 measurement device
210 acquisition unit
220 measurement unit

1. A learning device comprising a learning unit that performs learning related to an output of vital data indicating vital signs of a subject by using first sensor data acquired from the subject through a first method as learning data and using training data based on second sensor data acquired from the subject in the same period as a period of acquisition of the first sensor data through a second method which is less affected by noise than the first method,
wherein the learning unit performs learning further on the basis of third sensor data which is acquired in the same period as the period of acquisition of the first sensor data and the second sensor data and available as an index indicating a magnitude of influence of the noise occurring in the first sensor data.

2. The learning device according to claim 1, wherein the third sensor data includes acceleration data acquired with the subject or a device predicted to come into contact with the subject as a detection target, and
the learning unit performs learning based on the acceleration data as the third sensor data.

3. The learning device according to claim 2, wherein the learning unit performs learning based on at least the acceleration data in a direction of gravity as the third sensor data.

4. The learning device according to claim 1, wherein the vital data includes data related to cardiac activity, and
the learning unit learns an output of data related to cardiac activity of the subject by using a first electrocardiographic waveform acquired through the first method and the third sensor data acquired in the same period as a period of acquisition of the first electrocardiographic waveform as learning data and using training data based on a second electrocardiographic waveform acquired in the same period as the period of acquisition of the first electrocardiographic waveform through the second method.

5. The learning device according to claim 4, wherein the first method is a method of acquiring an electrocardiographic waveform using at least two electrodes expected to come into contact with the subject, and
the second method is a method of acquiring an electrocardiographic waveform using at least two electrodes attached to the skin of the subject.

6. The learning device according to claim 5, wherein the two electrodes used in the first method are provided on a seat on which the subject sits and an operated device which is operated by the subject.

7. The learning device according to claim 1, wherein the subject is a driver who drives a moving object.

8. A learning method comprising performing learning related to an output of vital data indicating vital signs of a subject by using first sensor data acquired from the subject through a first method as learning data and using training data based on second sensor data acquired from the subject in the same period as a period of acquisition of the first sensor data through a second method which is less affected by noise than the first method,
wherein the learning further includes performing learning further on the basis of third sensor data which is acquired in the same period as the period of acquisition of the first sensor data and the second sensor data and available as an index indicating a magnitude of influence of the noise occurring in the first sensor data.

9. A measurement device comprising a measurement unit that outputs vital data indicating vital signs of a subject using first sensor data acquired from the subject through a first method as an input,
wherein the measurement unit outputs the vital data by using the first sensor data as learning data, using training data based on second sensor data acquired from the subject in the same period as a period of acquisition of the first sensor data through a second method which is less affected by noise than the first method, and using a trained model having performed learning related to an output of the vital data further on the basis of third sensor data which is acquired in the same period as the period of acquisition of the first sensor data and the second sensor data and available as an index indicating a magnitude of influence of the noise occurring in the first sensor data.
